(19) United States
(12) Patent Application Publication
(10) Pub. No.: US 2025/0209615 A1
(43) Pub. Date: Jun. 26, 2025

(54) DETERMINING TRANSDUCER LOCATIONS FOR DELIVERY OF TUMOR TREATING FIELDS USING SIMULATIONS BASED ON APPROXIMATE TUMOR LOCATION

(71) Applicant: Novocure GmbH, Root D4 (CH)

(72) Inventors: Oren PELES ZEEVI, Haifa (IL); Amit FELDMAN, Haifa (IL)

(73) Assignee: Novocure GmbH, Root D4 (CH)

(21) Appl. No.: 18/960,626

(22) Filed: Nov. 26, 2024

Related U.S. Application Data
(60) Provisional application No. 63/613,858, filed on Dec. 22, 2023.

(51) Int. Cl. G06T 7/00; G06V 10/25; G16H 50/20

(52) U.S. Cl. CPC G06T 7/0012; G06V 10/25; G16H 50/20; G06T 2207/20081; G06T 2207/30096

(57) ABSTRACT

A method for determining transducer locations for delivery of tumor treating fields based on approximate abnormality location, includes: receiving a selection of a healthy model from a plurality of healthy models, the healthy model being representative of a subject; receiving an identification of a location of an abnormality and a size of the abnormality; associating, by at least one processor, a model of the abnormality with the healthy model based on the location of the abnormality and the size of the abnormality to obtain a customized model of the subject; receiving a selection of locations on the customized model of the subject to place transducers to treat the abnormality; calculating for each of the locations, a dosage of tumor treating fields treatment for a region of interest in the customized model of the subject; and selecting one or more of the locations as recommended locations based on the dosage.

### CROSS-REFERENCE TO RELATED APPLICATIONS

This Application claims priority to U.S. Provisional Application No. 63/613,858, filed Dec. 22, 2023, which is incorporated by reference herein in its entirety.

This Application is related to U.S. Provisional Application No. 63/613,835 filed Dec. 22, 2023, which is incorporated by reference herein in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1A depicts an example method for determining transducer locations for delivery of tumor treating fields using simulations based on approximate tumor location according to an embodiment.

FIG. 1B depicts an example method for generating healthy models according to an embodiment.

FIG. 2 depicts examples of healthy models.

FIG. 3 depicts an example plot used for clustering healthy models.

FIG. 4 depicts an example plot used for clustering healthy models.

FIG. 5 depicts an example plot of clusters of healthy models.

FIG. 6 depicts an example apparatus to apply alternating electric fields to a subject's body.

FIGS. 7A-7B illustrate schematic views of exemplary design of a transducer for applying alternating electric fields.

FIG. 8 depicts an example placement of transducers on a subject's head.

FIG. 9 depicts an example computer apparatus.

Various embodiments are described in detail below with reference to the accompanying drawings, wherein like reference numerals represent like elements.

### DESCRIPTION OF EMBODIMENTS

When generating transducer layouts for application of tumor treating fields (TTFields) for a subject, medical images of the subject are often used. Examples of such medical images are magnetic resonance imaging (MRI) scans, computed tomography (CT) medical image, positron emission tomography (PET) medical images, and/or the like including combinations and/or multiples thereof. Segmentation is often performed on medical images to divide a medical image into two or more segments with each segment representing a different object of interest. A medical image is often segmented to extract or isolate an object of interest (e.g., abnormal tissue) in the medical image from other structures (e.g., healthy tissue). As discovered by the inventors, performing segmentation on a medical image of a subject uses significant resources in terms of computing power and healthcare provider time. For example, to segment an image, the image is sliced, and an abnormality is segmented from healthy tissue in each slice, requiring significant time from the healthcare provider and significant computing resources to perform the segmentation. Moreover, segmentation on medical images of certain regions of the body are particularly computationally demanding. For example, medical images of a head typically show about five different types of tissues, while medical images of a torso show about twenty different types of tissues, making segmenting medical images of a torso significantly more computationally demanding than segmenting medical images of a head. Medical images of a torso are also significantly larger than medical images of a torso, for example, due to the relative size different in heads and torsos. Consequently, it may take a few minutes to segment a medical image of a head but upwards of an hour to segment a medical image of a torso.

One or more embodiments described herein provide for generating transducer layouts for application of TTFields for a subject without performing segmentation. In some embodiments, a healthy model may be selected from multiple healthy models, the selected healthy model being selected to represent a subject. For example, a healthy model with similar characteristics/properties (e.g., height, weight, body mass, gender, etc.) to the subject may be selected. A model of an abnormality (e.g., a tumor, a collapsed lung, liquid in the lung, a resection, an implanted device (e.g., a stent, an intravenous line or port, etc.)) for the subject may then be associated with the selected healthy model based on the size and location of the abnormality within the subject to create a customized model of the subject. The customized model to the subject may then be used to select locations to place transducers to treat the abnormality. A dosage of TTFields treatment is then calculated, for each of the locations, for a region of interest in the customized model of the subject. Thus, a subject-specific transducer layout is generated for the subject without segmenting medical images of the subject by using the healthy models and the model of the abnormality.

The embodiments described herein further provide a practical application of generating transducer layouts by avoiding the need to perform segmentation on medical images of the subject. By using the healthy models and model of the abnormality, the tissue conductivity is considered when generating transducer layouts for treating the subject with TTFields without having to perform segmentation on medical images of the subject. This approach yields more effective TTFields treatment for the subject while saving computational processing resources and healthcare provider time. Moreover, one or more embodiments described herein provide for generating transducer layouts specific to the subject where no medical images exist. These and other technical improvements may be realized using the one or more embodiments described herein.

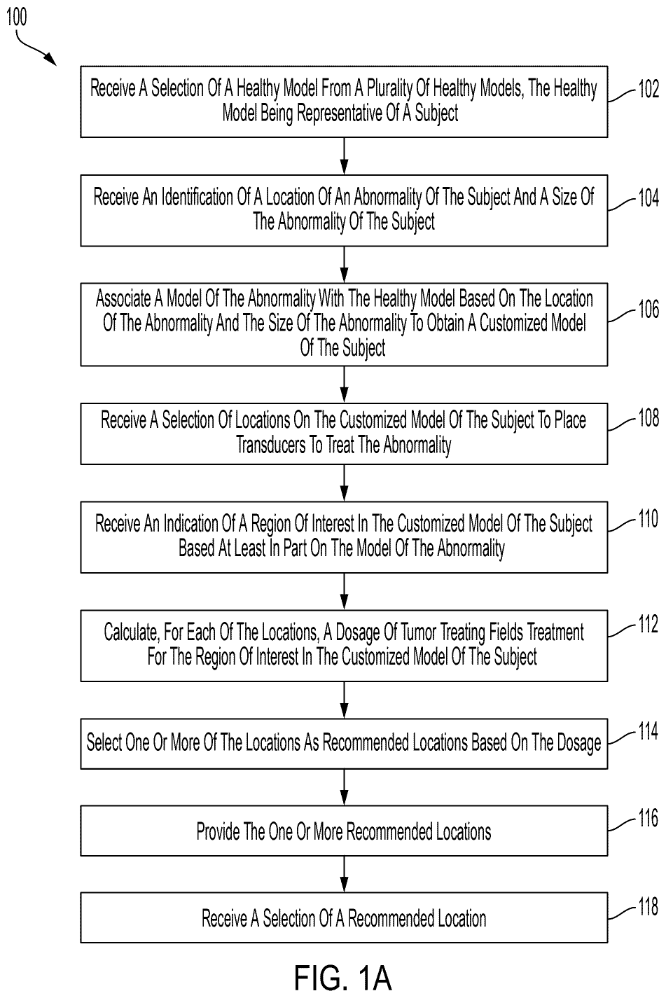

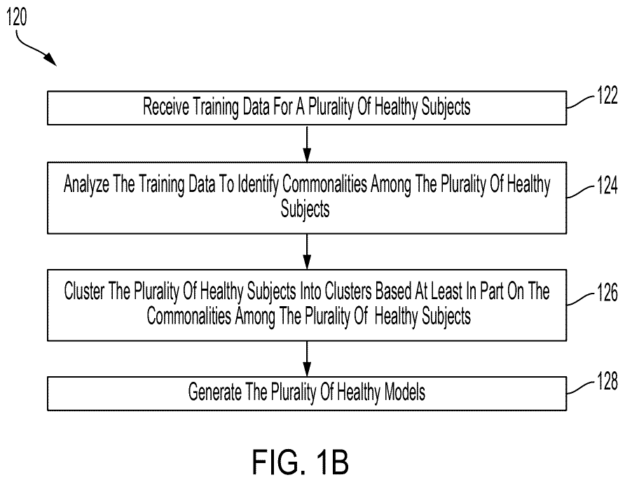

FIG. 1A depicts an example method for determining transducer locations for delivery of tumor treating fields using simulations based on approximate tumor location. Certain steps of the method 100 are described as computer-implemented steps. The computer may be, for example, any device comprising one or more processors and memory accessible by the one or more processors, the memory storing instructions that when executed by the one or more processors cause the computer to perform the relevant steps of the method 100. The method 100 may be implemented by any suitable system or apparatus, such as the apparatus 900 of FIG. 9. While an order of operations is indicated in FIG. 1A for illustrative purposes, the timing and ordering of such operations may vary where appropriate without negating the purpose and advantages of the examples set forth in detail herein.

With reference to FIG. 1A, at step 102, the method 100 includes receiving a selection of a healthy model from a plurality of healthy models, the healthy model being representative of a subject. In some embodiments, healthy models may be models of subjects known to be without or substantially without abnormalities in an area of interest (e.g., a head, torso, and/or the like including combinations and/or multiples thereof). For example, medical images of healthy subjects (e.g., those known to be without abnormalities in an area of interest) are captured and used to create healthy models of the healthy subjects. According to one or more embodiments described herein, the healthy model is selected from the plurality of healthy models based at least in part on a medical image of the subject (e.g., MRI image(s), CT image(s), PET image(s)), a physical measurement of the subject (e.g., a circumference of the subject's head or torso), a classification of the subject (e.g., a gender of the subject), and/or the like including combinations and/or multiples thereof. According to one or more embodiments described herein, the plurality of healthy models is based on healthy subjects clustered into groups. For example, the healthy models can be grouped based on characteristics of the healthy subjects from with the healthy models were created. The healthy models can be grouped according to any suitable characteristic(s), such as weight of the subject, height of the subject, body mass of the subject, gender of the subject, health information about the subject (e.g., previously surgical procedures, known diseases), and/or the like including combinations and/or multiples thereof. According to one or more embodiments described herein, each of the plurality of healthy models is representative of a group of healthy subjects without abnormalities. According to one or more embodiments described herein, each of the plurality of healthy models is segmented based on tissue type. The selection of the healthy model can be determined automatically (e.g., based on analyzing one or more medical images of the subject to identify similarities to one of the heathy models), manually (e.g., a healthcare provider can select a healthy model from the plurality of healthy models), and/or the like including combinations and/or multiples thereof. According to one or more embodiments described herein, the healthy models are generated according to the method 120 of FIG. 1B, which is now described in more detail.

In particular, FIG. 1B depicts an example method 120 for generating healthy models according to an embodiment. Certain steps of the method 120 are described as computer-implemented steps. The computer may be, for example, any device comprising one or more processors and memory accessible by the one or more processors, the memory storing instructions that when executed by the one or more processors cause the computer to perform the relevant steps of the method 120. The method 120 may be implemented by any suitable system or apparatus, such as the apparatus 900 of FIG. 9. While an order of operations is indicated in FIG. 1B for illustrative purposes, the timing and ordering of such operations may vary where appropriate without negating the purpose and advantages of the examples set forth in detail herein.

At step 122, the method 120 includes receiving training data for a plurality of healthy subjects. The training data includes information about the healthy subjects. For example, the training data can include medical history information (e.g., past diagnoses, past treatments), demographic information (e.g., height, weight, gender, body mass), medical images (e.g., MRI images, CT images), and/or the like including combinations and/or multiples thereof.

At step 124, the method 120 includes analyzing the training data to identify commonalities among the plurality of healthy subjects. Examples of commonalities include height, weight, gender, body mass, and/or the like including combinations and/or multiples thereof. According to one or more embodiments described herein, analyzing the training data includes performing a principal component analysis (PCA) to identify the commonalities among the plurality of healthy subjects. In other embodiments, other approaches to performing the commonality analysis can be performed.

At step 126, the method 120 includes clustering the plurality of healthy subjects into clusters based at least in part on the commonalities among the plurality of healthy subjects. For example, subjects that are similar in height, weight, gender, and body mass may be clustered into a cluster. According to one or more embodiments described herein, the clustering is performed using k-means clustering. In other embodiments, other approaches to cluster can be performed.

At step 128, the method 120 includes generating the plurality of healthy models. Generating the healthy models includes, for each cluster, generating one of the plurality of healthy models based at least in part on the training data for the plurality of healthy subjects that are within the cluster. According to an embodiment, a model of one of the plurality of healthy subjects within the cluster is selected to be the healthy model. According to another embodiment, the healthy model for the cluster is generated using information from two or more of the subjects within the cluster. According to one or more embodiments described herein, each of the healthy models is defined by a vector having a number of variables. The healthy model can be created from the variables of the healthy models of the healthy subjects within the cluster. As an example, the healthy model is an average of the variables of healthy models within the cluster.

With continued reference to FIG. 1A, at step 104, the method 100 includes receiving an identification of a location of an abnormality of the subject and a size of the abnormality of the subject.

At step 106, the method 100 includes associating, by at least one processor (e.g., the one or more processors 902 of FIG. 9), a model of the abnormality with the healthy model based on the location of the abnormality and the size of the abnormality to obtain a customized model of the subject. The location of the abnormality and the size of the abnormality can be determined automatically (e.g., based on analyzing one or more medical images of the subject), manually (e.g., a healthcare provider can determine the location of the abnormality and the size of the abnormality), and/or the like including combinations and/or multiples thereof. According to one or more embodiments described herein, the model of the abnormality is a simplified model of the abnormality. For example, the simplified model reduces the complexity of the model by omitting certain factors, making certain assumptions (e.g., about shape, size, orientation, etc.), and/or the like including combinations and/or multiples thereof. According to one or more embodiments described herein, the model of the abnormality is representative of an organ. For example, where the abnormality exists in an organ, such as a liver or a lung, the model can be a model of the organ (e.g., the liver or the lung). According to one or more embodiments described herein, the model of the abnormality is a geometric shape. For example, the model can be a two-dimensional shape (e.g., a circle, square, ellipse) or a three-dimensional shape (e.g., a sphere, a cube, an ellipsoid, a polygon, a concave shape, a convex shape), and/or the like including combinations and/or multiples thereof. According to one or more embodiments described herein, the model of the abnormality is based on a shape of the abnormality and the size of the abnormality. According to one or more embodiments described herein, associating the model of the abnormality with the healthy model includes defining electrical values of the abnormality for voxels of the healthy model that correspond to the location of the abnormality and the size of the abnormality. More particularly, electrical values associated with the abnormality are used in place of electrical values of the healthy model on a voxel-by-voxel basis. That is, electrical values for voxels in the healthy model associated with the model of the abnormality are substituted for electrical values representative of the abnormality. Such electrical values can include, for example, conductivity of tissue, such as tumorous tissue.

At step 108, the method 100 includes receiving a selection of locations on the customized model of the subject to place transducers to treat the abnormality. According to one or more embodiments described herein, the selection of locations on the customized model of the subject to place the transducers to treat the abnormality is based on the location of the abnormality with respect to the customized model. As an example, if the abnormality is in the head of the customized model, one or more of locations may include a first pair of transducers with a front-back relationship and a second pair of transducers with a left-right relationship (such as the transducer layout in FIG. 8). As an example, if the abnormality is in the torso of the customized model, one or more of locations may include a first pair of transducers with a front-back relationship and a second pair of transducers with a left-right relationship. According to one or more embodiments described herein, the selection of locations on the customized model of the subject to place the transducers to treat the abnormality is based at least in part on a preselected list of locations based on, for example, the healthy model and a general location of an abnormality (e.g., in the center of the head, in the front of the head, in the front left of the head, in the upper torso, in the upper left torso, etc.). The locations on the customized model of the subject can be selected automatically (e.g., based on analyzing one or more medical images of the subject), manually (e.g., a healthcare provider can select the locations), and/or the like including combinations and/or multiples thereof.

At step 110, the method 100 includes receiving an indication of a region of interest (ROI) in the customized model of the subject based at least in part on the model of the abnormality. According to one or more embodiments described herein, the region of interest is defined by a gross tumor volume (GTV) for the abnormality, which is then used to calculate the dosage of treatment (at step 112). The indication of the ROI can be determined automatically (e.g., based on analyzing one or more medical images of the subject to determine the ROI), manually (e.g., a healthcare provider can indicate the ROI), and/or the like including combinations and/or multiples thereof.

At step 112, the method 100 includes calculating, by the at least one processor (e.g., the one or more processors 902 of FIG. 9), for each of a plurality of locations, a dosage of TTFields treatment for the region of interest in the customized model of the subject. According to one or more embodiments described herein, calculating the dosage of TTFields treatment is based at least in part on the conductivities for the at least one tissue type included in the healthy model and the at least one tissue type included in the model of the abnormality. More particularly, the healthy model defines healthy tissue (e.g., non-tumorous tissue) and the model of the abnormality defines unhealthy tissue (e.g., tumorous tissue). The healthy tissue has a first electrical property and the unhealthy tissue has a second electrical property, and calculating the dosage of treatment is based at least in part on the first electrical property and the second electrical property. In this way, the dosage of TTFields treatment is based on the electrical properties of the abnormality (e.g., a tumor) and electrical properties of healthy tissues (e.g., tissues other than the abnormality). Calculating the dosage of TTFields treatment is further described in more detail in U.S. Patent Application Publication No. 2020/0023179, entitled “USING POWER LOSS DENSITY AND RELATED MEASURES TO QUANTIFY THE DOSE OF TUMOR TREATING FIELDS (TTFIELDS)” and U.S. Patent Application Publication No. 2021/0196943, entitled “METHODS, SYSTEMS, AND APPARATUSES FOR FAST APPROXIMATION OF ELECTRIC FIELD DISTRIBUTION,” the entire contents of both of which are incorporated by reference herein. It should be appreciated that these calculations in step 112 involve solving complex algorithms using large data sets associated with the healthy model and associated with the subject and, as such, require the use of a computer apparatus, as the human mind is not capable of performing the required calculations.

At step 114, the method 100 includes selecting one or more of the locations as recommended locations based on the dosage calculated in step 112. According to one or more embodiments described herein, the location with a highest dosage for the ROI may be selected as a recommended location. According to one or more embodiments described herein, the location with a second highest dosage for the ROI may be selected as a recommended location. According to one or more embodiments described herein, the location with a highest dosage for the ROI that also matches other criteria may be selected as a recommended location. As an example, other criteria to be considered may include: avoiding one or more avoidance areas of the subject (e.g., a nipple, a surgical scar, an eye, an ear, a mouth, a nose, irritated skin, a sensitive area, etc.); and providing maximized patient comfort. The recommended locations can be selected automatically (e.g., based on analyzing the dosages and the locations), manually (e.g., a healthcare provider can analyze the dosages and the locations), and/or the like including combinations and/or multiples thereof.

At step 116, the method 100 includes providing the one or more recommended locations selected in step 114. According to one or more embodiments described herein, each recommended location may be output as a representation of one or more pairs of transducers and locations for the one or more pairs of transducers on the subject. As an example, the representation of the subject may be based on the healthy model of the subject or the subject. As an example, a display is used to show a representation of the transducers and/or the locations on the subject. As an example, the output of the representation may be in form of a document.

At step 118, the method 100 includes receiving a selection of a recommended location. The recommended location can be selected automatically (e.g., based on analyzing one or more medical images of the subject), manually (e.g., a healthcare provider can select the recommended location from the possible locations), and/or the like including combinations and/or multiples thereof. The selected location can then be used to provide the dosage of TTFields treatment to the subject.

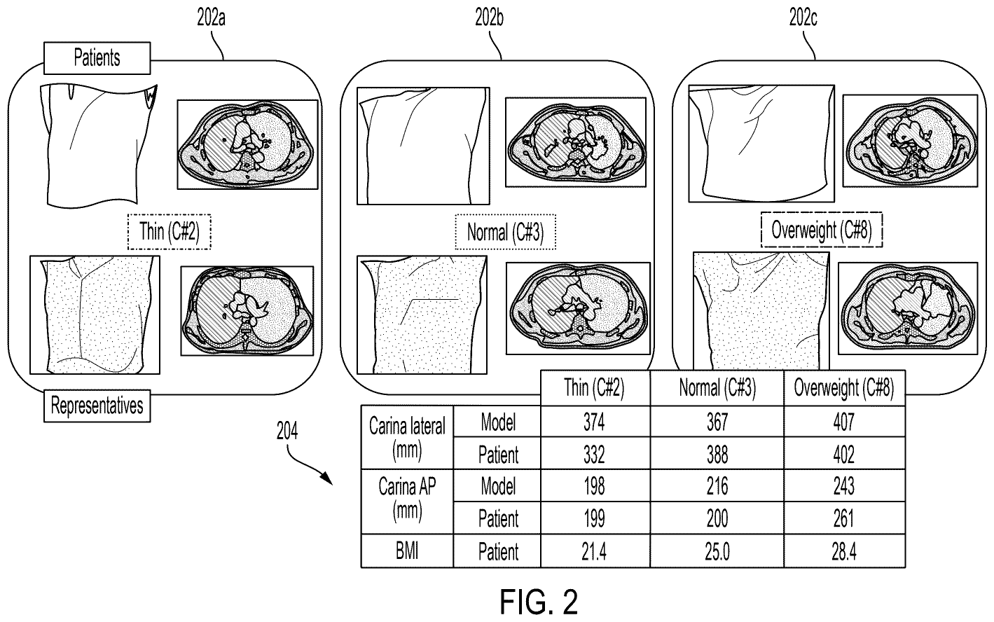

FIG. 2 depicts examples of healthy models 202a-202c. In this example, the healthy model 202a is a healthy model for a “thin” classification, the healthy model 202b is a healthy model for a “normal” classification, and the healthy model 202c is a healthy model for an “overweight” classification. As shown in the table 204, various values are associated with each of the healthy models 202a-202c, such as values for a carina lateral measurement, a carina anteroposterior (AP) measurement, and a body mass index. It should be appreciated that additional and/or alternative values can be associated with one or more of the healthy models 202a-202c in other embodiments. As can be seen in the table 204, corresponding values for the subject (e.g., “Patient”) are also shown.

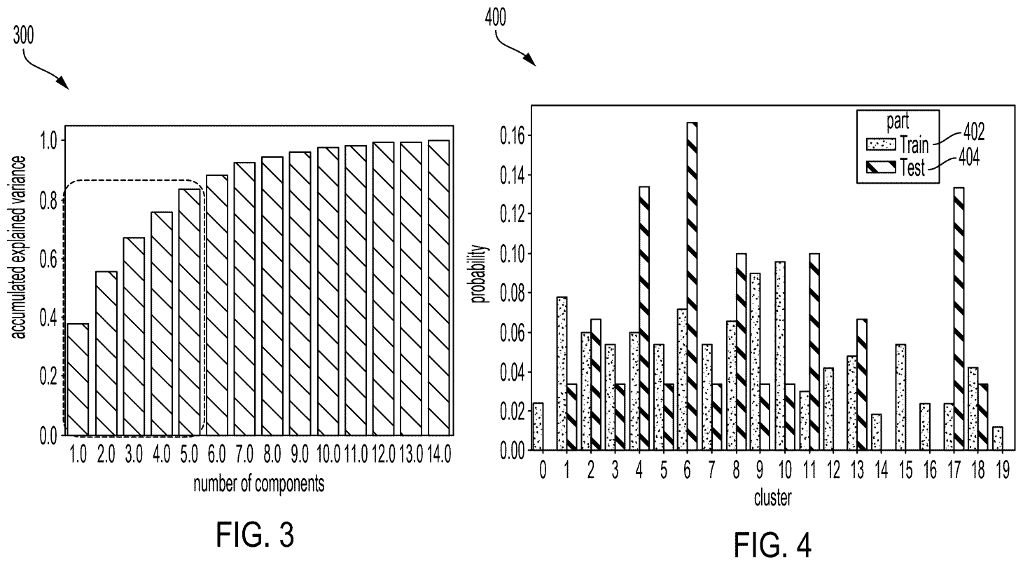

FIG. 3 depicts an example plot 300 used for clustering healthy models. In this example, a number of components are identified and plotted based on their accumulated explained variance. The components can be identified automatically (e.g., by a machine learning model trained to identify components from data of subjects), manually (e.g., selected by a subject matter expert), and/or combinations thereof. Non-limiting examples of components are: a carina anteroposterior (AP) measurement, a carina lateral measurement, a carina thoracic cavity lateral measurement a carina thoracic cavity lateral AP measurement, a carina mediastinum width measurement, a top liver AP measurement, a top liver lateral measurement, a top liver thoracic cavity lateral measurement, a top liver thoracic cavity AP measurement, a suprasternal notch AP measurement, a suprasternal notch lateral measurement, a suprasternal notch thoracic cavity lateral measurement, a suprasternal notch thoracic cavity AP measurement, a suprasternal notch to top liver measurement, and/or the like including combinations and/or multiples thereof. Principal component analysis can be used to identify a certain number of features as being relevant. For example, although fourteen components are used in the example of FIG. 3, it can be shown that the first five components account for 80% of the data and thus the remaining components can be ignored or discarded according to one or more embodiments described herein. In such cases, each healthy model would have a vector of five variables, each of the five variables corresponding to one of the five components.

FIG. 4 depicts an example plot 400 used for clustering healthy models. In this example, training data 402 and test data 404 are plotted for each of twenty clusters based on their respective probabilities. The training data 402 represents data about subjects that are used to train a machine learning model, for example, to perform clustering, and the test data 404 represents data about subjects used to test or verify the machine learning model after training. It should be appreciated that the training data 402 and the testing data 404 are different data according to one or more embodiments described herein.

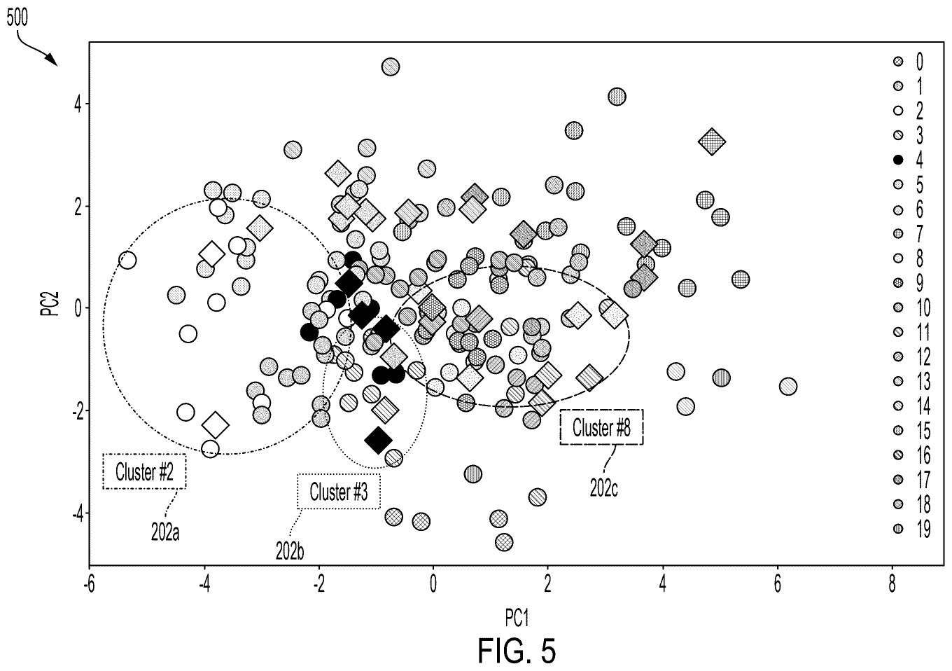

FIG. 5 depicts an example plot 500 of clusters of healthy models. In this example, data points for twenty different clusters are shown, corresponding to the plot 400. Three clusters 502a, 502b, 502c are shown, which correspond to the heathy models 202a, 202b, 202c, respectively, of FIG. 2. In the example of FIG. 5, data points for each cluster fall within a geometric shape, such as a circle or ellipse, as shown. According to one or more embodiments described herein, a centroid of the shape can be determined and can be used as the healthy model for that cluster. According to one or more embodiments described herein, one of the data points within the cluster can be selected to be the healthy model of the cluster.

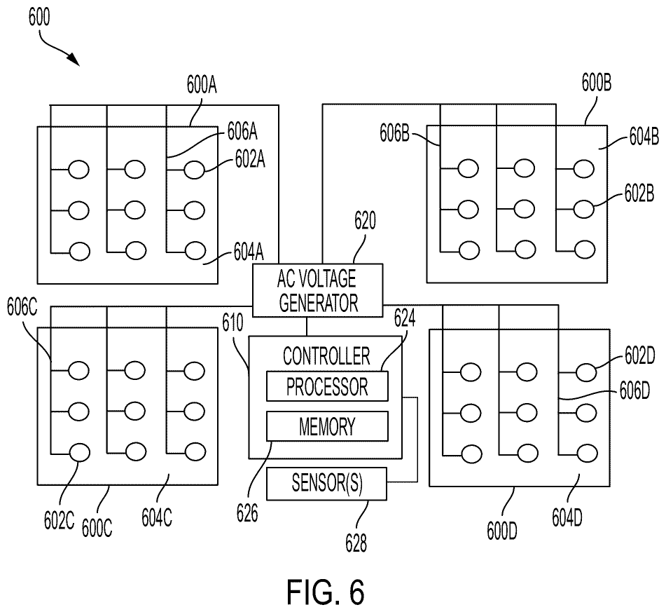

FIG. 6 depicts an example apparatus 600 to apply alternating electric fields (e.g., TTFields) to the subject's body. The system may be used for treating a target region of a subject's body with an alternating electric field. According to one or more embodiments described herein, at least two transducers are used to provide alternating electric fields, such as TTFields, to a subject. For example, one electrode element or multiple electrode elements can be used to provide the alternating electric fields, such as TTFields, to the subject. According to one or more embodiments described herein, one or more pairs of transducers are used to deliver alternating electric fields, such as TTFields, to the subject. In an example, the target region may be in the subject's brain, and an alternating electric field may be delivered to the subject's body via transducers (e.g., a pair of transducers, two pairs of transducer arrays, and/or the like including combinations and/or multiples thereof) positioned on a head of the subject's body (such as, for example, in FIG. 8, which has four transducers 800). In another example, the target region may be in the subject's torso, and an alternating electric field may be delivered to the subject's body via transducers (e.g., a pair of transducers, two pairs of transducer arrays, and/or the like including combinations and/or multiples thereof) positioned on at least one of a thorax, an abdomen, or one or both thighs of the subject's body. Other transducer array placements on the subject's body may be possible.

The example apparatus 600 depicts an example system having four transducers (or “transducer arrays”) 600A-D. It should be appreciated that other numbers of transducers can be used in other embodiments, such as two transducers or more than four transducers. Each transducer 600A-D may include substantially flat electrode elements 602A-D positioned on a substrate 604A-D and electrically and physically connected (e.g., through conductive wiring 206A-D). The substrates 604A-D may include, for example, cloth, foam, flexible plastic, and/or conductive medical gel. Two transducers (e.g., 600A and 600D) may be a first pair of transducers configured to apply an alternating electric field to a target region of the subject's body. The other two transducers (e.g., 600B and 600C) may be a second pair of transducers configured to similarly apply an alternating electric field to the target region.

The transducers 600A-D may be coupled to an AC voltage generator 620, and the system may further include a controller 610 communicatively coupled to the AC voltage generator 620. The controller 610 may include a computer having one or more processors 624 and memory 626 accessible by the one or more processors. The memory 626 may store instructions that when executed by the one or more processors control the AC voltage generator 620 to induce alternating electric fields between pairs of the transducers 600A-D according to one or more voltage waveforms and/or cause the computer to perform one or more methods disclosed herein. The controller 610 may monitor operations performed by the AC voltage generator 620 (e.g., via the processor(s) 624). One or more sensor(s) 628 may be coupled to the controller 610 for providing measurement values or other information to the controller 610.

In some embodiments, the voltage generation components may supply the transducers 600A-D with an electrical signal having an alternating current waveform at frequencies in a range from about 50 kHz to about 1 MHz and appropriate to deliver TTFields treatment to the subject's body

The electrode elements 602A-D may be capacitively coupled. In one example, the electrode elements 602A-D are ceramic electrode elements coupled to each other via conductive wiring 606A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 600A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating electric field.

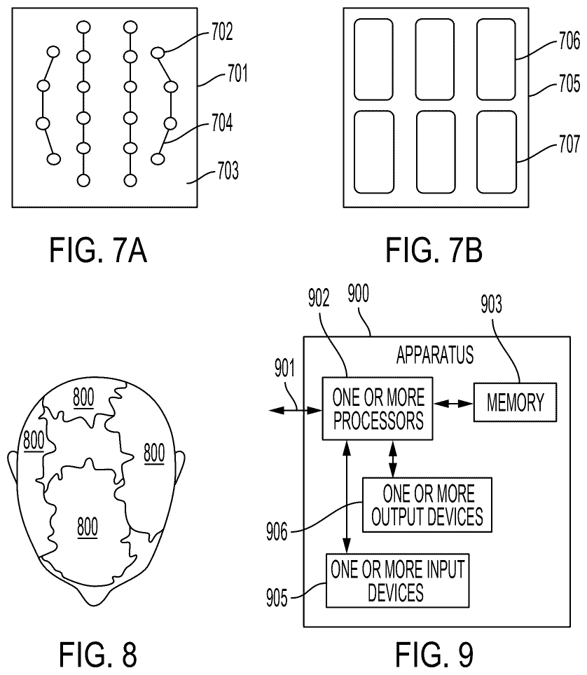

FIG. 7A illustrates a schematic view of an exemplary design of a transducer for applying alternating electric fields. Transducer 701 includes twenty electrode elements 702, which are positioned on substrate 703, and electrode elements 702 are electrically and physically connected to one another through a conductive wiring 704. In some embodiments, electrode elements 702 may include a ceramic disk.

FIG. 7B illustrates a schematic view of an exemplary design of a transducer for applying alternating electric fields. Transducer 705 may include substantially flat electrode elements 706. In some embodiments, electrode elements 706 are non-ceramic dielectric materials positioned over flat conductors. Examples of non-ceramic dielectric materials positioned over flat conductors may include polymer films disposed over pads on a printed circuit board or over substantially planar pieces of metal. In some embodiments, such polymer films have a high dielectric constant, such as, for example, a dielectric constant greater than 10. In some embodiments, electrode elements 706 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 706 may have a same shape, similar shapes, and/or different shapes.

FIG. 9 depicts an example computer apparatus for use with one or more embodiments described herein. As an example, apparatus 900 may be a computer to implement certain inventive techniques disclosed herein, such as determining transducer locations for delivery of TTFields based on approximate tumor location. As an example, method 100 of FIG. 1A and/or the method 120 of FIG. 1B may be performed by a computer, such as the apparatus 900. As an example, steps 102 to 118 of FIG. 1A and/or the steps 122 to 128 of FIG. 1B may be performed by a computer, such as apparatus 900. In some embodiments, controller 610 of FIG. 6 may be implemented with apparatus 900. As an example, some or all of the steps in the method illustrated in FIGS. 1A and 1B may be performed on a single apparatus 900. As an example, the steps in the method illustrated in FIGS. 1A and 1B may be performed by one, two, three, four, or more apparatuses 900. As an example, the apparatus 900 may be a portable computer device (e.g., a mobile device, a tablet or a laptop).

The apparatus 900 may include one or more processors 902, memory 903, one or more input devices 905, and one or more output devices 906.

Input to the apparatus 900 may be provided by one or more input devices 905, provided from one or more input devices in communication with the apparatus 900 via link 901 (e.g., a wired link or a wireless link; e.g., with a direct connection or over a network), and/or provided from another computer(s) in communication with the apparatus 900 via link 901.

Output for the apparatus 900 may be provided by one or more output devices 906, provided to one or more output devices in communication with the apparatus 900 via link 901, and/or provided from another computer(s) in communication with the apparatus 900 via link 901. The one or more output devices 906 may provide the status of the operation according to some embodiments described herein, such as transducer array selection, voltages being generated, and other operational information. The output device(s) 906 may provide visualization data according to some embodiments described herein. The one or more output devices 906 may include one more displays and one or more speakers. The output device(s) 906 may display healthy models, a model of the abnormality, a customized model, and/or recommended locations as described with respect to FIGS. 1A and 1B, and/or another suitable outputs associated with determining transducer locations for delivery of tumor treating fields based on approximate tumor location according to one or more embodiments described herein.

In some embodiments, one or more input devices 905 and one or more output devices 906 may be combined into one or more unitary input/output devices (e.g., a touch screen).

In some embodiments, based on input from one or more input devices 905 or input from outside the apparatus 900 via the link 901, the one or more processors 902 may perform operations as described herein. For example, based on input 901, the one or more processors 902 may generate control signals to control the voltage generator to implement one or more embodiments described herein. As an example, user input may be received from the one or more input devices 905. As an example, input may be from another computer in communication with the apparatus 900 via link 901. As an example, input may be from one or more input devices in communication with the apparatus 900 via link 901.

In some embodiments, the one or more processors 902 may perform operations as described herein and provide results of the operations as output. As an example, output may be provided to the one or more output devices 906. As an example, output may be provided to another computer in communication with the apparatus 900 via link 901. As an example, output may be provided to one or more output devices in communication with the apparatus 900 via link 901.

The memory 903 may be accessible by the one or more processors 902 so that the one or more processors 902 may read information from and write information to the memory 903. The memory 903 may store instructions that, when executed by the one or more processors 902, implement one or more embodiments described herein. The memory 903 may be a non-transitory computer readable medium (or a non-transitory processor readable medium) containing a set of instructions thereon for determining transducer locations for delivery of tumor treating fields based on approximate tumor location, wherein when executed by a processor (such as one or more processors 902), the instructions cause the processor to perform one or more methods discussed herein. As an example, the apparatus 900 may be a computer, and memory of the computer may store an app, a program, or a software to perform embodiments described herein.

The apparatus 900 may be an apparatus for determining transducer locations for delivery of tumor treating fields based on approximate tumor location, the apparatus including: one or more processors (such as one or more processors 902); and memory (such as memory 903) accessible by the one or more processors, the memory storing instructions that when executed by the one or more processors, cause the apparatus to perform one or more methods described herein.

The memory 903 may be a non-transitory processor readable medium containing a set of instructions thereon for determining transducer locations for delivery of tumor treating fields based on approximate tumor location, wherein when executed by one or more processors (such as one or more processors 902), the instructions cause the one or more processors to perform one or more methods described herein.

### ILLUSTRATIVE EMBODIMENTS

The invention includes other illustrative embodiments (“Embodiments”) as follows.

Embodiment 1. A method for determining transducer locations for delivery of tumor treating fields based on approximate abnormality location, the method comprising: receiving a selection of a healthy model from a plurality of healthy models, the healthy model being representative of a subject; receiving an identification of a location of an abnormality of the subject and a size of the abnormality of the subject; associating, by at least one processor, a model of the abnormality with the healthy model based on the location of the abnormality and the size of the abnormality to obtain a customized model of the subject; receiving a selection of locations on the customized model of the subject to place transducers to treat the abnormality; receiving an indication of a region of interest in the customized model of the subject based at least in part on the model of the abnormality; calculating, by the at least one processor, for each of the locations, a dosage of tumor treating fields treatment for the region of interest in the customized model of the subject; selecting one or more of the locations as recommended locations based on the dosage; and providing the one or more recommend locations.

Embodiment 2. The method of embodiment 1, wherein the model of the abnormality is a simplified model of the abnormality.

Embodiment. 3. The method of embodiment 1, wherein the model of the abnormality is representative of an organ.

Embodiment 4. The method of embodiment 1, wherein the model of the abnormality is a geometric shape.

Embodiment 5. The method of embodiment 1, wherein the model of the abnormality is based on a shape of the abnormality and the size of the abnormality.

Embodiment 5A. The method of embodiment 1, wherein the abnormality is a tumor.

Embodiment 6. The method of embodiment 1, wherein associating the model of the abnormality with the healthy model comprises defining electrical values of the abnormality for voxels of the healthy model that correspond to the location of the abnormality and the size of the abnormality.

Embodiment 7. The method of embodiment 1, wherein the region of interest defined by a gross tumor volume (GTV) for the abnormality, wherein the dosage of treatment is calculated based at least in part on the GTV.

Embodiment 8. The method of embodiment 1, wherein the selection of locations on the customized model of the subject to place the transducers to treat the abnormality is based at least in part on conductivities for at least one tissue type included in the healthy model and at least one tissue type included in the model of the abnormality.

Embodiment 9. The method of embodiment 8, wherein calculating the dosage of tumor treating fields treatment is based at least in part on the conductivities for the at least one tissue type included in the healthy model and the at least one tissue type included in the model of the abnormality.

Embodiment 10. The method of embodiment 1, wherein the healthy model defines healthy tissue and the model of the abnormality defines unhealthy tissue, wherein the healthy tissue has a first electrical property and the unhealthy tissue has a second electrical property, wherein calculating the dosage of treatment is based at least in part on the first electrical property and the second electrical property.

Embodiment 11. The method of embodiment 1, wherein each of the plurality of healthy models is segmented based on tissue type.

Embodiment 11A. The method of embodiment 1, wherein each of the plurality of healthy models is representative of a group of healthy subjects without abnormalities.

Embodiment 11B. The method of embodiment 1, wherein the plurality of healthy models is based on healthy subjects clustered into groups.

Embodiment 12. The method of embodiment 1, wherein the plurality of healthy models are generated by: receiving training data for a plurality of healthy subjects; analyzing the training data to identify commonalities among the plurality of healthy subjects; clustering the plurality of healthy subjects into clusters based at least in part on the commonalities among the plurality of healthy subjects; and generating the plurality of healthy models, wherein the generating comprises, for each cluster, generating one of the plurality of healthy models based at least in part on the training data for the plurality of healthy subjects that are within the cluster.

Embodiment 13. The method of embodiment 12, wherein generating the plurality of healthy models comprises selecting, for each cluster, a model of one of the plurality of healthy subjects within the cluster to be the healthy model.

Embodiment 14. The method of embodiment 12, wherein generating the plurality of healthy models comprises generating, for each cluster, the healthy model using information concerning at least two of the plurality of healthy subjects within the cluster.

Embodiment 15. The method of embodiment 12, wherein analyzing the training data comprises performing a principal component analysis to identify the commonalities among the plurality of healthy subjects.

Embodiment 15A. The method of embodiment 12, wherein the clustering is performed using k-means clustering.

Embodiment 15B. The method of embodiment 1, wherein each of the healthy models is defined by a vector having a number of variables.

Embodiment 16. The method of embodiment 1, wherein the healthy model is selected from the plurality of healthy models based at least in part on a medical image of the subject.

Embodiment 17. The method of embodiment 1, wherein the healthy model is selected from the plurality of healthy models based at least in part on a physical measurement of the subject.

Embodiment 18. The method of embodiment 1, wherein the healthy model is selected from the plurality of healthy models based at least in part on a classification of the subject.

Embodiment 19. An apparatus for determining transducer locations for delivery of tumor treating fields based on approximate abnormality location, the apparatus comprising: one or more processors; and a memory accessible by the one or more processors, the memory storing instructions that when executed by the one or more processors, cause the apparatus to: receive a selection of a healthy model from a plurality of healthy models, the healthy model being representative of a subject; receive an identification of a location of an abnormality of the subject and a size of the abnormality of the subject; associate a model of the abnormality with the healthy model based on the location of the abnormality and the size of the abnormality to obtain a customized model of the subject; receive a selection of locations on the customized model of the subject to place transducers to treat the abnormality; receiving an indication of a region of interest in the customized model of the subject based at least in part on the model of the abnormality; calculate, for each of the locations, a dosage of tumor treating fields treatment for the region of interest in the customized model of the subject; selecting one or more of the locations as recommended locations based on the dosage; and providing the one or more recommended locations.

Embodiment 20. A non-transitory processor readable medium containing a set of instructions thereon for determining transducer locations for delivery of tumor treating fields using simulations based on approximate abnormality location, wherein when executed by a processor, the instructions cause the processor to: receive a selection of a healthy model from a plurality of healthy models, the healthy model being representative of a subject; receive an identification of a location of an abnormality of the subject and a size of the abnormality of the subject; associate a model of the abnormality with the healthy model based on the location of the abnormality and the size of the abnormality to obtain a customized model of the subject; receive a selection of locations on the customized model of the subject to place transducers to treat the abnormality; receive an indication of a region of interest in the customized model of the subject based at least in part on the model of the abnormality; calculate, for each of the locations, a dosage of abnormality treating fields treatment for the region of interest in the customized model of the subject; selecting one or more of the locations as recommended locations based on the dosage; and providing the one or more recommended locations.

Embodiment 21. A method, machine, manufacture, and/or system substantially as shown and described.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A method for determining transducer locations for delivery of tumor treating fields based on approximate abnormality location, the method comprising:
receiving a selection of a healthy model from a plurality of healthy models, the healthy model being representative of a subject;
receiving an identification of a location of an abnormality of the subject and a size of the abnormality of the subject;
associating, by at least one processor, a model of the abnormality with the healthy model based on the location of the abnormality and the size of the abnormality to obtain a customized model of the subject;
receiving a selection of locations on the customized model of the subject to place transducers to treat the abnormality;
receiving an indication of a region of interest in the customized model of the subject based at least in part on the model of the abnormality;
calculating, by the at least one processor, for each of the locations, a dosage of tumor treating fields treatment for the region of interest in the customized model of the subject;
selecting one or more of the locations as recommended locations based on the dosage; and
providing the one or more recommended locations.

2. The method of claim 1, wherein the model of the abnormality is a simplified model of the abnormality.

3. The method of claim 1, wherein the model of the abnormality is representative of an organ.

4. The method of claim 1, wherein the model of the abnormality is a geometric shape.

5. The method of claim 1, wherein the model of the abnormality is based on a shape of the abnormality and the size of the abnormality.

6. The method of claim 1, wherein associating the model of the abnormality with the healthy model comprises defining electrical values of the abnormality for voxels of the healthy model that correspond to the location of the abnormality and the size of the abnormality.

7. The method of claim 1, wherein the region of interest defined by a gross tumor volume (GTV) for the abnormality, wherein the dosage of treatment is calculated based at least in part on the GTV.

8. The method of claim 1, wherein the selection of locations on the customized model of the subject to place the transducers to treat the abnormality is based at least in part on conductivities for at least one tissue type included in the healthy model and at least one tissue type included in the model of the abnormality.

9. The method of claim 8, wherein calculating the dosage of tumor treating fields treatment is based at least in part on the conductivities for the at least one tissue type included in the healthy model and the at least one tissue type included in the model of the abnormality.

10. The method of claim 1, wherein the healthy model defines healthy tissue and the model of the abnormality defines unhealthy tissue, wherein the healthy tissue has a first electrical property and the unhealthy tissue has a second electrical property, wherein calculating the dosage of treatment is based at least in part on the first electrical property and the second electrical property.

11. The method of claim 1, wherein each of the plurality of healthy models is segmented based on tissue type.

12. The method of claim 1, wherein the plurality of healthy models are generated by:
receiving training data for a plurality of healthy subjects;
analyzing the training data to identify commonalities among the plurality of healthy subjects;
clustering the plurality of healthy subjects into clusters based at least in part on the commonalities among the plurality of healthy subjects; and
generating the plurality of healthy models, wherein the generating comprises, for each cluster, generating one of the plurality of healthy models based at least in part on the training data for the plurality of healthy subjects that are within the cluster.

13. The method of claim 12, wherein generating the plurality of healthy models comprises selecting, for each cluster, a model of one of the plurality of healthy subjects within the cluster to be the healthy model.

14. The method of claim 12, wherein generating the plurality of healthy models comprises generating, for each cluster, the healthy model using information concerning at least two of the plurality of healthy subjects within the cluster.

15. The method of claim 12, wherein analyzing the training data comprises performing a principal component analysis to identify the commonalities among the plurality of healthy subjects.

16. The method of claim 1, wherein the healthy model is selected from the plurality of healthy models based at least in part on a medical image of the subject.

17. The method of claim 1, wherein the healthy model is selected from the plurality of healthy models based at least in part on a physical measurement of the subject.

18. The method of claim 1, wherein the healthy model is selected from the plurality of healthy models based at least in part on a classification of the subject.

19. An apparatus for determining transducer locations for delivery of tumor treating fields based on approximate abnormality location, the apparatus comprising:
one or more processors; and
a memory accessible by the one or more processors, the memory storing instructions that when executed by the one or more processors, cause the apparatus to:
receive a selection of a healthy model from a plurality of healthy models, the healthy model being representative of a subject;
receive an identification of a location of an abnormality of the subject and a size of the abnormality of the subject;
associate a model of the abnormality with the healthy model based on the location of the abnormality and the size of the abnormality to obtain a customized model of the subject;
receive a selection of locations on the customized model of the subject to place transducers to treat the abnormality;
receiving an indication of a region of interest in the customized model of the subject based at least in part on the model of the abnormality;
calculate, for each of the locations, a dosage of tumor treating fields treatment for the region of interest in the customized model of the subject;
select one or more of the locations as recommended locations based on the dosage; and
provide the one or more recommended locations.

20. A non-transitory processor readable medium containing a set of instructions thereon for determining transducer locations for delivery of tumor treating fields using simulations based on approximate abnormality location, wherein when executed by a processor, the instructions cause the processor to:
receive a selection of a healthy model from a plurality of healthy models, the healthy model being representative of a subject;
receive an identification of a location of an abnormality of the subject and a size of the abnormality of the subject;
associate a model of the abnormality with the healthy model based on the location of the abnormality and the size of the abnormality to obtain a customized model of the subject;
receive a selection of locations on the customized model of the subject to place transducers to treat the abnormality;
receive an indication of a region of interest in the customized model of the subject based at least in part on the model of the abnormality;
calculate, for each of the locations, a dosage of abnormality treating fields treatment for the region of interest in the customized model of the subject;
select one or more of the locations as recommended locations based on the dosage; and
provide the one or more recommended locations.
